(19) United States
(12) Patent Application Publication
(10) Pub. No.: US 2005/0085454 A1
(43) Pub. Date: Apr. 21, 2005

(54) Phenolic antioxidant-chromium complexes for treatment or prevention of type 2 diabetes or glucose intolerance

(75) Inventor: Shibnath Ghosal, Calcutta (IN)

Correspondence Address: DR. WALTER KATZ, 8 UMBERLAND PLACE, MONROE TOWNSHIP, NJ, 08831, US

(73) Assignee: NATREON INC.

(21) Appl. No.: 10/686,801

(22) Filed: Oct. 16, 2003

(51) Int. Cl. A61K 31/555; A61K 31/28

(52) U.S. Cl. 514/185; 514/505

(57) ABSTRACT

A composition for the treatment, prevention or management of a condition in primates, especially humans comprising a phenolic antioxidant-chromium complex.

### CROSS-REFERENCE TO RELATED PATENTS AND APPLICATIONS

This invention is related to U.S. Pat. Nos. 6,124,268; 6,440,436; and 6,558,712; issued to the same inventor as herein and Disclosure Document No. 525805 dated Feb. 11, 2003.

### BACKGROUND OF THE INVENTION

1. Field of the Invention

This invention relates to a phenolic antioxidant-chromium complex for treating, preventing or maintaining a condition in primates, especially humans, particularly Type 2 diabetes or glucose intolerance, and more particularly, it relates to chromium complexed with agents which are low molecular weight hydrolyzable tannins of plant origin and/or purified Shilajit containing oxygenated dibenzo-α-pyrone (DBP) or its conjugates, including dimers and oligomers and fulvic acids, obtained by extraction of native Shilajit, and pharmaceutical and nutritional compositions thereof, useful for supplementing dietary chromium, lowering blood glucose and serum lipid, including the lowering of undesirably high blood serum LDL-cholesterol levels and the raising of blood serum HDL-cholesterol levels and increasing lean body mass.

2. Description of the Prior Art

Diabetes is a human condition which is actually a group of diseases characterized by high levels of blood glucose resulting from deficits in insulin production, insulin action, or both. The most common forms of diabetes are Type 1 and Type 2 diabetes. Type 1, which typically strikes children and young adults, occurs when the body's immune system destroys pancreatic β-cells, which are the only cells that synthesize insulin, the substance that regulates blood glucose levels.

Type 2 diabetes, or non-insulin dependent diabetes mellitus (NIDDM), is more commonly associated with advanced age, obesity, family history, physical inactivity, and even race/ethnicity. This diabetes disease usually begins as insulin resistance, a disorder in which the cells fail to use insulin properly. In this condition, when the need for insulin arises, the pancreas gradually loses its ability to synthesize insulin. Most products that attempt to control diabetes target the glucose level in the body.

Type 2 diabetes (NIDDM), is increasingly common throughout the world. The World Health Organization has predicted that between 1997 and 2025, the number of diabetics will double from 143 million to about 300 million. The incidence of NIDDM is highest in economically developed nations, particularly the U.S., where approximately 6.5% of the population (17 million people) have either diagnosed or undiagnosed diabetes. The leading cause of mortality and morbidity in people with NIDDM is cardiovascular disease caused by macro- and microvascular degeneration. Current therapies for NIDDM focus primarily on weight reduction. Indeed, several investigations indicate that 65% to 75% of cases of diabetes in Caucasians could be avoided if individuals in this subgroup did not exceed their ideal weight. The success of this approach has been, at best, modest. An alternate approach to the control of Type 2 diabetes is to arrest the progress of the pathology until a cure has been found. To this end, some investigators suggest that dietary antioxidants may be of value. Several studies in humans and laboratory animals with NIDDM indicate that vitamin E and lipoic acid supplements lessen the impact of oxidative damage caused by dysregulation of glucose metabolism.

There is mounting evidence that a general increase in antioxidant status achieved by dietary supplementation can help diminish oxidative stress associated with NIDDM. Certain antioxidants are of particular benefit with regard to the prevention and treatment of diabetic complications. Primary among these are vitamin E (-tocopherol) and lipoic acid (thioctic acid). Vitamin E is a fat-soluble vitamin that effectively scavenges the peroxyl radical in cell membranes, thereby inhibiting lipid peroxidation. Prospective epidemiological studies demonstrate that high serum vitamin E levels are associated with a decreased risk of NIDDM. In the GK rat, a model for NIDDM, Vitamin E supplementation has significantly improved glycemic control, possibly by minimizing free radical damage to the pancreatic β-cells. Improvements in glucose metabolism and insulin action in the obese Zucker rat, an animal that exhibits many of the features of NIDDM, may be mediated by a reduction in oxidative stress. Researchers found that glucose-stimulated hyper-insulinemia and lipid peroxidation in the obese Zucker rat could be significantly reduced with dietary Vitamin E. A similar finding has been observed in humans. Plasma concentrations of lipid hydroperoxides, an indicator of lipid peroxidation, were higher in healthy, insulin-resistant volunteers as compared to insulin-sensitive ones, while plasma concentrations of Vitamin E were significantly lower. A placebo-controlled explorative study of patients with NIDDM indicated that oral administration of lipoic acid significantly increased insulin-mediated glucose uptake, presumably by modulating insulin sensitivity.

A number of other antioxidant nutrients have been reported to be beneficial for subjects with NIDDM. Flavonoids, a group of antioxidant polyphenolic compounds, found ubiquitously in commonly consumed fruits and vegetables, and in beverages, such as red wine and tea, have been demonstrated to protect against oxidative stress in Type 1 and Type 2 diabetes. Specifically, flavonoids can inhibit lipid oxidation and delay the depletion of lipid-soluble antioxidants. Serum levels of carotenoids, another group of antioxidant compounds often present in edible plants, were inversely related to fasting serum insulin levels. While not conclusive, this observation is suggestive of a role for carotenoids in the pathogenesis of insulin resistance and diabetes. Taurine and coenzyme Q₁₀ are endogenous antioxidants that can also be obtained from the diet. In rats with diabetes induced by chemical destruction of β-cells, taurine supplementation (1% taurine in the drinking water) reduced renal oxidant injury by decreasing lipid peroxidation and inhibiting the accumulation of advanced glycation end products within the kidney. The effects of oral treatment with coenzyme Q₁₀ (60 mg twice daily) were examined in a randomized, double-blind trial of 30 patients with coronary heart disease. After 8 weeks of treatment, patients receiving coenzyme Q₁₀ had reduced plasma levels of insulin (fasting and 2-hr), glucose, and lipid peroxides, as compared to the control group. These findings indicate that treatment with coenzyme Q₁₀ in this group decreases oxidative stress and improves insulin sensitivity.

Chromium is an essential trace element involved in the metabolism of carbohydrates, lipids and proteins, primarily by increasing the efficiency of insulin production. Chromium deficiency affects the maintenance of normal glucose tolerance and healthy lipid profiles. The estimated requirement for chromium in humans is about 1 μg/day, but only 1 to 3% of trivalent chromium is absorbed. In the USA, chromium intakes range from 20 to 50 μg/day, with plasma levels from 0.05 to 0.5 μg/L (1.0 to 9.6 nmole/L). The Food and Nutrition Board of the NAS/NRC states that a safe and adequate intake of chromium for an adult is 50 to 200 μg/day. However, the dietary intake of chromium in humans is often suboptimal. Chromium assessment has proven to be a challenge due to the low amounts of chromium present in biological materials and the absence of a reliable indicator of chromium level in the body. Recently, chromium has been touted as an agent for increasing lean body mass and decreasing percent body fat.

Chromium exists mostly in two valence states in nature, namely, hexavalent chromium [chromium(VI)] and trivalent chromium [chromium(III)]. Chromium(VI) is commonly used in industrial chrome plating, welding, painting, metal finishes, steel manufacturing, alloy, cast iron and wood treatment, and is a proven toxin, mutagen and carcinogen. The mechanistic cytotoxicity of chromium(VI) is not completely understood; however, many studies have demonstrated that chromium(VI) induces oxidative stress, DNA damage, apoptotic cell death and altered gene expression. Conversely, chromium(III) is essential for proper insulin function and is required for normal protein, fat and carbohydrate metabolism, and is acknowledged as a dietary supplement. Chromium(III), in absence of antioxidants, is converted to Chromium(VI) by spontaneous systematic oxidation hence it induces delayed toxicity. This can be avoided using Chromium(III) complexed with an appropriate antioxidant-ligand. Comparative concentration- and time-dependent effects of chromium(VI) and chromium(III) have demonstrated an increased production of reactive oxygen species (ROS) and lipid peroxidation, enhanced excretion of urinary lipid metabolites, DNA fragmentation and apoptotic cell death in both in vitro and in vivo models. Chromium(VI) demonstrated significantly higher toxicity as compared with chromium(III). Chromium(VI) induced more pronounced oxidative damage in multiple target organs in p53 deficient mice.

Comparative studies of chromium(III) picolinate and niacin-bound chromium(III), two popular dietary supplements, reveal that chromium(III) picolinate produces significant oxidative stress and DNA damage. Studies have implicated the toxicity of chromium picolinate in renal impairment, skin blisters and pustules, anemia, hemolysis, tissue edema, liver dysfunction; neuronal cell injury, impaired cognitive, perceptual and motor activity; enhanced production of hydroxyl radicals, chromosomal aberration, depletion of antioxidant enzymes, and DNA damage. Recently, chromium picolinate has been shown to be mutagenic with the picolinic acid moiety apparently responsible because studies show that picolinic acid alone is clastogenic. Niacin-bound chromium(III) has been demonstrated to be more bioavailable and efficacious, and no toxicity has been reported for this complex. In summary, these studies demonstrate that a cascade of cellular events including oxidative stress, genomic DNA damage and modulation of apoptotic regulatory gene p53 are involved in chromium(VI)-induced toxicity and carcinogenesis, and that the safety of chromium(III) is largely dependent on its ligand.

Despite forty years of research on the potential role of chromium in carbohydrate and lipid metabolism, significant progress has only recently been made regarding the mode of action of chromium at the molecular level. The oligopeptide low-molecular-weight chromium-binding substance (LMWCr) has been shown to function as part of a novel insulin-signaling autoamplification mechanism. The proposed mechanism of action for this substance also sheds some light on the potential of chromium-containing compounds as nutritional supplements or in the treatment of adult-onset diabetes and other conditions.

The U.S. Recommended Daily Intake (RDI) of chromium is 120 μg. U.S. Pat. Nos. 5,087,623, 5,087,624; and 5,175,156; the entire contents of which are hereby incorporated by reference, described the administration of such an effective amount of chromic tripicolinate for the treatment of adult-onset diabetes. International Patent Application No. WO96/35421 disclosed the use of high doses of chromic tripicolinate (providing 1,000-10,000 μg chromium/day) for reducing hyperglycemia and stabilizing the level of serum glucose in humans with Type II diabetes.

Nicotinic acid and picolinic acid form coordination complexes with monovalent, divalent and trivalent metal ions and facilitate the absorption of these metals by transporting them across intestinal cells and into the bloodstream. Chromium absorption in rats following oral administration of CrCl₃ was facilitated by the non-steroidal anti-inflammatory drugs (NSAIDs) aspirin and indomethacin. These drugs inhibit the enzyme cyclooxygenase which converts arachidonic acid to various prostaglandins, resulting in inhibition of intestinal mucus formation and lowering of intestinal pH which facilitates chromium absorption.

### OBJECTS OF THE INVENTION

Accordingly, an object of this invention is to provide a composition and method for treating, preventing or maintaining Type 2 diabetes or glucose intolerance in primates, especially humans, that employs a safe and effective phenolic antioxidant-chromium complex, without pro-oxidation activity, while providing a beneficial effect to the blood profile.

A further object of this invention is to provide an orally delivered composition useful for treating or preventing Type 2 diabetes in primates, especially humans.

A further object of this invention is to provide a phenolic antioxidant-chromium complex for treating primates, especially humans with Type 2 diabetes or impaired glucose tolerance, which leads to an improvement in blood glucose, insulin and lipid variables, reduction in glycosylated hemoglobulin and diabetic retionopathy, and an improvement in pancreatic islet cell regeneration.

A further object of this invention is to administer a phenolic antioxidant-chromium complex to primates, especially humans as a prophylactic or therapeutic agent for controlling various blood serum parameters. In particular, the administration is for controlling blood serum lipid levels, including the lowering of undesirably high blood serum LDL-cholesterol levels and the raising of blood serum HDL-cholesterol levels.

Other objects and features of this invention will be made apparent to one skilled in the art upon reading the following specification and claims.

### SUMMARY OF THE INVENTION

What is described herein is a composition for the treatment, prevention or management of a condition in primates, especially humans which comprises a phenolic antioxidant-chromium complex, preferably, Type 2 diabetes or non-insulin dependent diabetes mellitus, or glucose intolerance.

The invention also provides for compositions of phenolic antioxidant and niacin- or picolinic acid-bound chromium. These compositions are preferably comprised of dosage units effective to reduce cholesterol levels, such as about 100-500 mg of phenolic antioxidant per day and about 2-10 mg of niacin- or picolinic acid-bound chromium per day.

In one embodiment of the invention the composition includes a phenolic antioxidant of plant origin having no pro-oxidation activity, wherein phenolic antioxidant(s) include low molecular weight hydrolyzable tannins having a molecular weight below 2,000, preferably below 1,000, which can be obtained from the genus Phyllanthus, Terminalia, Gardenia, Geranium, Erodium or Tamarix, for example, from Phyllanthus emblica (syn. Emblica officinalis) fruit, Phyllanthus amarus plant, Phyllanthus flexusus plant and other Phyllanthus species, Terminalia bellerica and other Terminalia species, Erodium pelagonium, Geranium thumbergi plant, Tamarix aphyla or other Tamarix species.

In another embodiment of the invention, the composition of the invention comprises chromium complex(es) of oxygenated dibenzo-α-pyrone (DBP) or its conjugates, including dimers and oligomers and fulvic acids, which are suitable for the treatment, prevention or management of Type 2 diabetes or glucose tolerance in primates, especially humans.

Suitably, the oxygenated dibenzo-α-pyrone (DBP) or its conjugates, including dimers and oligomers and fulvic acids are obtained from purified Shilajit, described in the aforementioned U.S. Pat. No. 6,124,268.

Accordingly, a typical composition of the invention comprises chromium complex(s) of antioxidant fractions, e.g. of the Phyllanthus emblica plant and/or purified Shilajit.

Suitably, the chromium content in the complex is about 0.01 to 20% of the complex, preferably 0.02 to 10% and, most preferably 1 to 8% of the complex, preferably wherein the chromium is trivalent in nature.

The phenolic antioxidant-chromium complex of the invention is prepared by reacting a trivalent chromium salt with a phenolic antioxidant(s), for example, by reacting chromium chloride, acetate or formate because their anions do not materially affect the chemistry and stability of the complex, with a phenolic antioxidant(s) in an aqueous system. Preferably the reaction is carried out with low molecular weight tannins having a molecular weight below 2,000, most preferably below 1,000, or with oxygenated dibenzo-α-pyrone (DBP) or its conjugates, including dimers and oligomers and fulvic acids of purified Shilajit, or mixtures thereof.

The final composition phenolic antioxidant-chromium complex composition is obtained by spray, freeze, tray or vacuum drying.

Alternately, finely powdered chromium chloride, acetate, formate, or chromium picolinate or nicotinate or polynicotinate is dry blended with phenolic antioxidant to provide the phenolic antioxidant-chromium complex composition of the invention.

The invention herein includes formulations wherein the phenolic antioxidant-chromium complex is combined with a pharmaceutically or nutritionally acceptable excipients for the treatment of Type 2 diabetes or glucose tolerance in primates, especially humans.

In another aspect of the invention, the composition herein can also include a suitable added active ingredient, for example, an antioxidant, vitamin, carnitine, carnosine, N-acetyl-L-cysteine, aminoguanidine, polycosanol, a fatty acid or plant extract, and mixtures thereof.

### DETAILED DESCRIPTION OF THE INVENTION COMPLEX OF THE INVENTION

1. Chromium

A. Sources of Chromium

The chromium constituent of the complex of this invention is Cr³⁺, not Cr⁶⁺, for reasons described above. Cr³⁺ can be furnished effectively from any chromium salt, preferably, chromium chloride, chromium acetate or chromium formate compounds. These chromium compounds are preferred as starting materials for the preparation of the invention complex, because their anions do not materially affect the stability of the complex, and are commercially available compounds. Alternately, chromium picolinate or chromium nicotinate or chromium polynicotinate can be used as a source of Cr³⁺.

2. Phenolic Antioxidant(s)

A. Selection of Phenolic Antioxidants

The phenolic antioxidant(s) of the complex herein can chelate transition metals, e.g. chromium, iron and copper, which ordinarily, in the presence of hydrogen peroxide, will catalyze the formation of the biologically detrimental hydroxyl radical from superoxide anion radicals present endogenously in the body. However, transition metal-catalyzed formation of such hydroxyl radicals from a superoxide anion radical and hydrogen peroxide requires the availability of at least one coordination site in the transition metal that is either free or occupied by a readily dissociable ligand, such as water. This coordination with water may be completely displaced by stronger ligands. Accordingly, the phenolic antioxidants selected herein are capable of occupying all the available coordination sites in chromium, thereby eliminating the possibility of forming an undesirable oxo-chromium complex.

B. Sources of Selected Phenolic Antioxidants

In this invention, the selected phenolic antioxidants are low molecular-weight hydrolyzable tannins obtained by extraction from tannin-rich plants, (see U.S. Pat. No. 6,124,268); and/or oxygenated dibenzo-α-pyrone (DBP) or its conjugates, including dimers and oligomers and fulvic acids, obtained by extraction of native Shilajit, (see U.S. Pat. No. 6,440,436). Other suitable sources of low molecular-weight tannins are described by T. Okuda et al, “Hydrolyzable Tannins and Related Polyphenols”, in Fortsc. Chem. Org. Naturst, 66, 1-117 (1995), such tannin-containing plants include Phyllanthus emblica (syn. Emblica officinalis), Phyllanthus amarus, Phyllanthus flexusus and other Phyllanthus species, Terminalia bellerica and other Terminalia species, Erodium pelagonium, Geranium thumbergi, Tamarix aphyla and other Tamarix species.

3. Phenolic Antioxidant-Chromium Complex of Invention

A. Preparation of the Complex (Solution Method)

The complex of the invention can be prepared from a solution of a chromium salt (chromium chloride, acetate or formate) in an appropriate amount of water. Typically, to the intense green-colored solution is added a suitable excess amount of the phenolic antioxidant with rapid stirring. A suitable ratio of chromium ion to the phenolic antioxidant is about 1:4 to 1:10000, preferably 1:9 to 1:5000, most preferably 1:10 to 1:100, wherein complete engagement of the Cr³⁺ ion is achieved with excess phenolic antioxidant which forms association with the complex and thereby interact optimally with biological tissues, while keeping the chromium ion in its +3 state.

Production of a blackish-green colored opaque solution indicates the formation of the desired complex. Then this solution can be evaporated to dryness to provide the complex as a green-colored solid. Alternately, the solution containing the complex can be filtered and then dried.

B. Optical Detection of the Complex

Two absorbance maxima ordinarily are observed for CrCl₃. 6H₂O in the visible region, namely, at 622 nm and 436 nm. However, the Cr³⁺ phenolic antioxidant complex of the invention, e.g. wherein the phenolic fraction is the extract of the Phyllanthus emblica plant, has only one absorption maximum at 584 nm with reduced optical density value compared to the 622 nm of CrCl₃.6H₂O.

Similarly, two absorbance maxima are observed for Cr(HCOO)₃, in the visible region at 436 nm and 582 nm, whereas the Cr³⁺ complex with the phenolic fraction of Phyllanthus emblica has only one absorption maximum at 574 nm.

C. Preparation of the Complex (Dry Blending)

The complex of the invention can be prepared by dry blending a fine powder of any chromium salt (preferably, chromium chloride, acetate or formate) or suitable chromium complex (e.g. chromium picolinate or nicotinate or polynicotinate) with a phenolic antioxidant (preferably, Phyllanthus emblica and/or purified Shilajit) in a suitable blender.

4. Pharmaceutical and Nutritional Formulations of the Complex of the Invention

A. Preparation of Formulations

Pharmaceutical and nutritional formulations of the phenolic antioxidant-chromium complex of the invention may include suitable pharmaceutical and/or nutritional excipient(s) that are suitable for oral administration. Generally, these oral formulations of the invention fall into one of five categories:
1. A solution, suspension or syrup that is ready for oral administration, or,
2. A dry powder composition that can be combined with water just prior to use, i.e., a reconstitutable composition, or
3. A liquid concentrate ready for dilution prior to administration, or
4. A tablet ready for oral administration, or
5. A capsule ready for oral administration.

The orally administered vehicle in these formulations normally has no therapeutic activity and is nontoxic, but presents the active constituent to the body tissues in a form appropriate for absorption. Suitable absorption of the complex normally will occur most rapidly and completely when the composition is presented as an aqueous solution. However, modification of the vehicle with water-miscible liquids or substitution with water-immiscible liquids can affect the rate of absorption. Preferably, the vehicle of greatest value for the present inventive composition is water that meets the USP specification for water for injection. Generally, water of suitable quality for compounding will be prepared either by distillation or reverse osmosis to meet these USP specifications. The appropriate specifications for such formulations are given in Remington: The Science and Practice of Pharmacy, 19th Ed. at pp. 1526-1528. In preparing formulations which are suitable for oral administration, one can use aqueous vehicles, water-miscible vehicles, or non-aqueous vehicles. Water-miscible vehicles are also useful in the formulation of the composition of this invention. The most important solvents in this group are ethyl alcohol, polyethylene glycol, and propylene glycol.

Another useful formulation is a reconstitutable composition which is a sterile solid packaged in a dry form. The reconstitutable dry solid is usually packaged in a sterile container with a butyl rubber closure to ensure the solid is kept at an optimal moisture range. A reconstitutable dry solid is formed by dry filling, spray drying, or freeze-drying methods. See Pharmaceutical Dosage Forms: Parenteral Medications, 1, pp. 215-227.

Additional substances may be included in the compositions of this invention to improve or safeguard the quality of the composition. Thus, an added substance may affect solubility, provide for patient comfort, enhance the chemical stability, or protect preparation against the growth of microorganisms. The composition also may include an appropriate solubilizer, or substances which act as antioxidants, and a preservative to prevent the growth of microorganisms. These substances will be present in an amount that is appropriate for their function, and will not adversely affect the action of the composition. Appropriate antioxidants are found in Remington at pp. 1529. Examples of suitable antimicrobial agents include thimerosal, benzethonium chloride, benzalkonium chloride, triclosan, methyl p-hydroxybenzoate, propyl p-hydroxybenzoate, and parabens.

Preferred pharmaceutical or nutritional formulations are those suitable for oral administration to warm-blooded animals.

The compositions herein may contain the complex ingredient alone, or in combination with a pharmaceutically or nutritionally acceptable excipient, in dosage unit forms such as tablets, coated tablets, hard or soft gelatin capsules or syrups. These administratable forms can be prepared using known procedures, for example, by conventional mixing, granulating, tablet coating, dissolving or lyophilisation processes. Thus, pharmaceutical or nutritional compositions for oral administration can be obtained by combining the active ingredient with solid carriers, optionally granulating the resulting mixture, and processing the mixture by granulation, if desired or necessary, after the addition of suitable excipients, to give tablets or coated tablet cores.

Suitable excipients are, in particular, fillers, such as sugars, for example, lactose, sucrose, mannitol or sorbitol; cellulose preparations and/or calcium phosphates, for example, tricalcium phosphate or calcium hydrogen phosphate; and binders, such as starches, for example, corn, wheat, rice or potato starch, gelatin, tragacanth, methyl cellulose and/or polyvinylpyrrolidone, and/or, if desired, disintegrants, such as the above mentioned starches, and also carboxymethyl starch, cross-linked polyvinylpyrrolidone, agar, alginic acid or a salt thereof such as sodium alginate, and/or flow regulators and lubricants, for example, silica, talc, stearic acid or salts thereof such as magnesium stearate or calcium stearate, and/or polyethylene glycol. Coated tablet cores can be provided with suitable coatings, which if appropriate are resistant to gastric juices, using, inter alia, concentrated sugar solutions which may contain gum arabic, talc, polyvinylpyrrolidone, polyethylene glycol and/or titanium dioxide, shellac solutions in suitable organic solvents or solvent mixtures or, for the preparation of coatings resistant to gastric juices, solutions of suitable cellulose preparations such as acetylcellulose phthalate or hydroxypropylmethylcellulose phthlate. Dyes or pigments can be added to the tablets or coated tablets, for example, to identify or indicate different doses of the active complex ingredient.

Other pharmaceutical or nutritional preparations suitable for oral administration are hard gelatin capsules and also soft gelatin capsules made from gelatin and a plasticizer such as glycerol or sorbitol. Hard capsules may include the inventive complex in admixture with fillers such as lactose, binders such as starches, and/or lubricants such as talc or magnesium stearate, and if desired, stabilizers. In soft capsules, the inventive complex is preferably dissolved or suspended in a suitable liquid, such as fatty oil, paraffin oil or a liquid polyethylene glycol, to which a stabilizer can be added.

Phenolic antioxidant-chromium complex, when obtained by dry blending process, converts into true complex when formulated in aqueous or alcoholic systems. Alternately, this dry blended material can get converted into an effective complex when administered to primate, especially human.

B. Other Active Ingredients

The formulations of the invention may include an added active ingredient other than the complex itself, including:
(1) Antioxidants: e.g. Alpha lipoic acid (lowers cholesterol, protects LDL against oxidation and beneficial and treating Type 2 diabetes), Coenzyme Q (enhances beta cell function and glycemic control), Vitamin C (lowers blood glucose levels, inhibits glycation, prevents accumulation of sorbitol), Vitamin E (reduces oxidative stress, enhances insulin sensitivity).
(2) Other Vitamins: e.g. Biotin (aids in metabolism of macronutrients, enhances glucose utilization and is beneficial in diabetic neuropathy), Niacin (reduces blood glucose levels).
(3) Carnitine (improves blood glucose management and HbAlc levels, increases insulin sensitivity and glucose storage).
(4) Carnosine (opposes glycation).
(5) N-acetyl-L-cysteine (which protects beta-cells form free-radical damage).
(6) Aminoguanidine (assists in controlling cross-linking, a process that would advance diabetic complications).
(7) Policosanol (a mixture of essential alcohols from sugar cane wax—saccharaum officinarium; assist in lowering LDL-C and total cholesterol, and in increasing HDL-C)
(8) Fatty Acids: e.g. essential fatty acids (protect the plasma membrane), linolenic acid (aids weight loss and improves insulin sensitivity).
(9) Plant extracts: e.g. American ginseng (lowers blood glucose levels), Bilberry (reduces blood glucose), Ginkgo biloba (increases glucose-stimulated pancreatic beta-cell function), Garlic and Onions (hypoglycemic in nature).

The invention will now be described with particular reference to the following examples.

### EXAMPLE 1

### Preparation of Phenolic Antioxidants From Natural Sources

The following examples illustrate certain preferred embodiments and aspects of the present invention and are not to be construed as limiting the scope thereof.

A. Preparation of Phenolic Antioxidants from Phyllanthus emblica (syn. Emblica officinalis)

Fresh Emblica officinalis fruit (5 kg) was finely pulped and mixed with water (2:1) containing sodium chloride (1% w/w). The mixture was left standing at room temperature for about 12 hours. Then the mixture was stored in the cold (10° C.) for 3 days. Thereafter it was filtered through a thin cloth and the filtrate was spray-dried to obtain the tannin-fraction.

Standardization of the complex was controlled analytically by HPLC so that it contained at least 30% by weight of low molecular-weight hydrolyzable tannins, preferably 50-75%.

B. Preparation of Phenolic Antioxidants from Phyllanthus emblica (syn. Emblica officinalis)

Fresh Emblica officinalis fruit juice was obtained by crushing the whole fruit and filtering off the pulp. The juice was immediately dried to a powder by spray or freeze-drying to provide the desired tannin rich-fraction. If needed, the juice can be filtered or centrifuged to remove water-insoluble material.

C. Preparation of Phenolic Antioxidants from

Other Plants (e.g., Phyllanthus amarus, Terminalia bellerica)

Plants producing small and medium molecular-weight gallo-ellagi (hydrolyzable) tannoids, were obtained by the following process.

Extraction of the freshly harvested plant (e.g. seeds, fruits and leaves) with hot (45 to 60° C.) aqueous (or containing 1% sodium chloride) hydroalcoholic (water-ethyl alcohol 60:40) was followed by centrifugation (discarding the precipitate) and drying (spray, freeze or tray) of the solvent-soluble fraction provided the desired tannin-rich extract as an amorphous powder.

Low and medium molecular weight gallo-ellagi (hydrolyzable) tannoids were detected in the extract by reverse phase HPLC. This analytical technique permits an estimation of the extent of oligomerization of the gallo-hexahydroxydiphenic acid (HHDP) moieties, and is based on the observed retention time, which increased with the extent of oligomerization (determined by authentic reference materials). Some labile oligomers, e.g. from Phyllanthus amarus (fruits) were isolated and purified, for further characterization, by centrifugal partition chromatography (CPC), which did not require any solid support and therefore, does not affect the integrity of the labile oligomeric tannoids.

D. Preparation of Phenolic Antioxidants from Native Shilajit (U.S. Pat. No. 6,440,436)

Purified shilajit compositions were obtained by an extraction procedure from native shilajit rock exudates, as follows:
(a) powdering native shilajit exudates and dissolving it in water as solvent,
(b) filtering the mixture to remove insoluble substances,
(c) evaporating water from the filtrate to obtain a brown viscous residue,
(d) extracting the residue with a hot organic solvent, e.g. methanol, to obtain both a soluble fraction which includes low molecular-weight bioactive phenolic compounds particularly oxygenated dibenzo-.alpha.-pyrones, and insoluble shilajit humic substances,
(e) adding dilute aqueous NaOH to the insoluble shilajit humic portion to precipitate polymeric quinones,
(f) acidifying the filtrate below a pH of about 3 to precipitate humic acids leaving a brown acidic solution of fulvic acids,
(g) fractionating said acidic solution by passing it over activated carbon to provide a solution of low-to-medium molecular-weight fulvic acids,
(h) passing the fulvic acid solution through a H⁺ion-exchange resin to concentrate the fulvic acids in solution,
(i) evaporating the solution, and
(j) combining the low-to-medium molecular-weight fulvic acids Mw 700-2000, with the low molecular-weight bioactive phenolic compounds in a suitable proportion, e.g. 9 to 5:1 by weight.

Standardization of such purified shilajit composition was controlled analytically so that the composition contained, by weight, (a) at least 0.3% oxygenated dibenzo-α-pyrones including mono- and dimers of 3,8-dihydroxy-dibenzo-α-alpha-pyrones (in free and/or conjugated forms); and (b) low-to-medium molecular-weight fulvic acids (Mw 700-2000) in an amount of at least 30%, preferably 50-70%.

### EXAMPLE 2

### Preparation of Phenolic Antioxidant-Chromium Complexes

The following examples also illustrate certain preferred embodiments and aspects of the present invention and are not to be construed as limiting the scope thereof.

A. Preparation of Phenolic Antioxidant-Chromium Complex

A solution of 513 mg of CrCl₃. 6H₂O was prepared in 100 ml distilled water with rapid stirring, resulting in an intense green-colored solution. Then 2500 mg of the tannin-rich fraction of Phyllanthus emblica extract (Example 1) was added to the green solution with stirring. The mixture was heated to about 40° C. for 30 min. Alternately, the mixture was stirred at room temperature (about 25° C.) for about 60 min. A blackish-green colored opaque solution was obtained. The solution of the complex was evaporated to dryness and a green-colored solid was recovered. Alternately, the solution of the complex was filtered and then evaporated to dryness. Alternately, the solution may be granulated with a filler, such as, microcrystalline cellulose to form a granulation of the complex which can be filled into capsules or pressed into tablets after further processing.

1. Spectral Properties of Complex

The complex had an absorption maximum at 584 nm, which did not shift even after the addition of a sodium azide solution, indicating the absence of any free coordination sites in the complex.

2. Antioxidant Activity

An ABTS [(2,2′-Azinobis (3-ethylbenzthiazoline-6-sulfonic acid)] radical cation decolorizing assay was used to determine the superoxide scavenging activity of the complex. The IC₅₀ of the complex was found to be 2.46 μg/ml, indicating that the complex is an excellent superoxide quencher.

A diphenyl picrylhydrazide (DPPH) assay was used to determine the nitrogen radical quenching ability of the complex. An antioxidant activity of 88% at 40 μg/ml indicated that the complex is an excellent antioxidant. B. Alternate Preparation of Phenolic Antioxidant-Chromium Complex

The process described above was repeated using chromium formate in place of CrCl₃. 6H₂O.

The absorption maximum was 574 nm; the azide shift test was negative; the antioxidant ABTS activity IC₅₀ was 3.99 μg/ml; and the DPPH activity was 90% at 40 μg/ml.

C. Additional Preparations of Phenolic Antioxidant-Chromium Complex

1. 0.20% Cr in the Complex

The process described in A. above was repeated using 2.588 g of Chromium Chloride (containing 0.5052 g of Cr), 250 ml distilled water, and 250 g of the tannin-rich fraction of Phyllanthus emblica extract (Example 1). The resultant solid upon drying was yellowish-brown colored instead of the green-colored solid obtained in the process described in A. above.

2. 5.68% Cr in the Complex

The process described in A. above was repeated using 102.5 g of Chromium Chloride (containing 20 g of Cr), 250 ml distilled water, and 250 g of the tannin-rich fraction of Phyllanthus emblica extract (Example 1). The resultant green-colored solid obtained had a crystalline structure.

3. 10.00% Cr in the Complex

The process described in A. above was repeated using 105 g of Chromium Chloride (containing 20.50 g of Cr), 250 ml distilled water, and 100 g of the tannin-rich fraction of Phyllanthus emblica extract (Example 1). The resultant green-colored solid obtained had a soft sheet-like structure.

4. 2.00% Cr in the Granulation

A 5.68% Cr in the complex was prepared as in C. 2. above. A granulation was made using 80% of the solution [200 g of tannin-rich fraction of Phyllanthus emblica extract +82 g of chromium chloride (containing 16 g of Cr) in complex form] and 518 g of Microcrystalline Cellulose in a KitchenAid® mixer (Model: K45SSWH) for 35 minutes. The granulation was tray dried in an oven pre-set at 50° C. and a green-colored solid (powder and lumps) was recovered. This was milled and remixed to assure content uniformity.

### EXAMPLE 3

### Preparation of Pharmaceutical and Nutritional Formulations of Phenolic Antioxidant-Chromium Complex of Invention

The following examples illustrate certain preferred embodiments and aspects of the present invention and are not construed as limiting the scope thereof.

A. TABLETS AND CAPSULES OF THE INVENTIVE
COMPLEX

| | | Quantity per | |
|---|---|---|---|
| Ingredient | | Tablet/Capsule | |
| 1. | Chromium (from Inventive Complex) | 50.00-500.00 | mcg |
| 2. | Avicel pH 101 | 200.00 | mg |
| 3. | Starch 1500 | 189.00 | mg |
| 4. | Stearic acid, N.F. (powder) | 8.50 | mg |
| 5. | Cab-O-Sil | 2.00 | mg |

Note: The target weight of tablet/capsule is 400 mg; Avicel pH 101
and Starch may be adjusted suitably to reach the target weight.
The blended material can be filled into appropriate capsules.

B. ANTI-DIABETIC SUPPORT TABLETS/CAPSULES
OF THE INVENTIVE COMPLEX

| | | Quantity per | |
|---|---|---|---|
| Ingredient | | Tablet/Capsule | |
| 1. | Chromium (from Inventive Complex) | 50.00-500.00 | mcg |
| 2. | Vitamin B-6 (as Pyridoxine HCl) | 10 | mg |
| 3. | L-Arginine | 50 | mg |
| 4. | L-Lysine Monohydrochloride | 50 | mg |

| | | |
|---|---|---|
| 5. | Cellulose | q.s. |
| 6. | Magnesium stearate | q.s. |
| 7. | Gelatin | q.s. |

C. CARDIO-VASCULAR SUPPORT TABLETS
OF THE INVENTIVE COMPLEX

| | | Quantity per | |
|---|---|---|---|
| Ingredient | | Tablet | |
| 1. | Chromium (from Inventive Complex) | 50.00-500.00 | mcg |
| 2. | Vitamin A (Beta Carotene) | 45,000 | IU |
| 3. | Vitamin B-1 (Thiamin) | 50 | mg |
| 4. | Inositol Hexanicotinate | 500 | mg |
| 5. | Vitamin B-6 (Pyridoxine HCL) | 25 | mg |
| 6. | Vitamin B-12 (Cyanocobalamin) | 500 | mcg |
| 7. | Folic Acid | 800 | mcg |
| 8. | Vitamin C (Magnesium Ascorbate) | 1,500 | mg |
| 9. | Vitamin E D-alpha Tocophery (Natural) | 400 | IU |
| 10. | Copper (Sebacate) | 750 | mcg |
| 11. | Magnesium (Ascorbate, Taurinate, and | 300 | mg |
| | Oxide) | | |
| 12. | Potassium (Citrate) | 99 | mg |
| 13. | Selenium (L-Selenomethionine) | 200 | mcg |
| 14. | Silica (from 400 mg of Horsetail Extract) | 28 | mg |

Other Ingredients and Herbs:

| | | | |
|---|---|---|---|
| 15. | Coenzyme Q10 (Ubiquinone) | 60 | mg |
| 16. | L-Carnitine L-Tartrate | 500 | mg |
| 17. | Hawathorn Berry Extract | 400 | mg |
| 19. | Grape Seed Extract | 100 | mg |
| 20. | L-Proline | 500 | mg |
| 21. | L-Lysine (HCL) | 500 | mg |
| 22. | N-Acetyl Glucosamine | 500 | mg |
| 23. | Bromelain (2,000 GDU per g) | 1,200 | mg |
| 24. | Taurine (Magnesium Taurinate) | 500 | mg |
| 25. | Inositol (Hexanicotinate) | 50 | mg |

D. MULTI-VITAMIN AND MINERAL SUPPLEMENT
TABLETS OF THE INVENTIVE COMPLEX

| | | Quantity per | |
|---|---|---|---|
| Ingredient | | Tablet | |
| 1. | Chromium (from Inventive Complex) | 50.00-500.00 | mcg |
| 2. | Vitamin A (beta carotene) | 25,000 | IU |
| 3. | Vitamin A (palmitate) | 10,000 | IU |
| 4. | Vitamin B-1 (Thiamin Nitrate) | 100 | mg |
| 5. | Vitamin B-2 (Riboflavin) | 100 | mg |
| 6. | Inositol Hexanicotinate, Niacinamide & | 200 | mg |
| | Niacin | | |
| 7. | Vitamin B-5 (Calcium D-Pantothenate) | 100 | mg |
| 8. | Vitamin B-6 ((Phyridoxine HCL) | 100 | mg |
| 9. | Vitamin B-12 (Cyanocobalamin) | 200 | mcg |
| 10. | Biotin | 500 | mcg |
| 11. | Folic Acid | 800 | mcg |
| 12. | Vitamin C | 1,800 | mg |
| | (Magnesium, Manganese & Zinc Ascorbates) | | |
| 13. | Fat-Soluble Vitamin C | 200 | mg |
| | (from 476 mg of Ascorbyl Palmitate) | | |
| 14. | Vitamin D-3 (Cholecalciferol) | 400 | IU |
| 15. | Vitamin E D-alpha Tocopheryl (Natural) | 600 | IU |
| 16. | Boron (Amino Acid Chelate) | 2 | mg |
| 17. | Calcium (Succinate, Carbonate, Malate) | 200 | mg |
| 18. | Copper (Sebacate) | 1 | mg |
| 19. | Iodine (from Kelp) 150 mcg, | 150 | mcg |
| | Magnesium (Ascorbate, Oxide, Succinate) | | |
| 20. | Manganese (Ascorbate) | 300 | mg |
| 21. | Molybdenum (Amino Acid Chelate) | 300 | mcg |
| 22. | Potassium (Succinate, alpha-Ketoglutarate) | 90 | mg |
| 23. | Selenium | 250 | mcg |
| | (L-Selenomethionine & Sodium Selenite) | | |
| 24. | Zinc (Zinc Monomethionine & Ascorbate) | 30 | mg |

Other Ingredients and Plant antioxidants: N-Acetyl Cysteine,
Succinic Acid (Free Form), Choline (Bitartrate), Inositol
(Hexanicotinate and Inositol), N-Acetyl Glucosamine, DMAE
(Bitartrate), N-Acetyl L-Tyrosine, Coenzyme Q10, Alpha-Lipoic
Acid, Quercetin, Milk Thisle Seed Extract, Grape Seed Extract,
Ginkgo Biloba, Bilberry Extract.

E. WEIGHT LOSS SUPPORT TABLETS OF THE
INVENTIVE COMPLEX

| | | Quantity per | |
|---|---|---|---|
| Ingredient | | Tablet/Capsule | |
| 1. | Chromium (from Inventive Complex) | 50.00-500.00 | mcg |
| 2. | Garcinia Cambogia Extract | 600 | mg |
| 3. | Bitter Orange Peel Standardized Extract | 165 | mg |
| 4. | Green Tea | 100 | mg |
| 5. | Cayenne | 150 | mg |
| 6. | Mustard Seed | 100 | mg |
| 7. | Ginger Root | 100 | mg |
| 8. | Piper nigrum | 100 | mg |
| 9. | Acetyl L-Carnitine | 100 | mg |
| 10. | Niacinamide | 50 | mg |
| 11. | Vitamin B-6 (Pyridoxine HCL) | 25 | mg |

F. ORAL LIQUID OF THE INVENTIVE COMPLEX

| | | Quantity per |
|---|---|---|
| Ingredient | | 100 ml |
| 1. | Chromium (from Inventive Complex)* | 1-10 mg |
| 2. | Purified Water | q.s. |
| 3. | Excipients: Preservatives, stabilizers, | q.s. |
| | sweetners, flavors, colors, etc. | |

Note: Quantity per serving size of 5 ml: 50.00-500.00 mcg

G. ORAL LIQUID (ADMINISTERED IN-SITU)
OF THE INVENTIVE COMPLEX

| | | Quantity per | |
|---|---|---|---|
| Ingredient | | 100 ml | |
| 1. | Chromium chloride* (CrCl₃.6H₂O) | 5-50 | mg |
| 2. | Phenolic antioxidant | 80 | mg |

| | | |
|---|---|---|
| 3. | Purified Water | q.s. |
| 4. | Excipients: Preservatives, stabilizers, | q.s. |
| | sweeteners, flavors, colors, etc. | |

Note: Quantity of chromium per serving size of 5 ml: 50.00-500.00 mcg

H. SNACKBAR WITH THE INVENTIVE COMPLEX

| Ingre- | | |
|---|---|---|
| dient | | Quantity per |
| No. | Ingredient | 1 Kg |
| 1 | Chromium (from Inventive Complex) | 50.00-500.00 mcg |
| 2 | Nutrition Blend: Calcium (Tricalcium | q.s |
| | Phosphate and Calcium Carbonate), | |
| | Magnesium (Magnesium Oxide), Vitamin | |
| | A, Vitamin C, Vitamin D-3, Vitamin B-1 | |
| | (Thiamin), Vitamin B-2 (Riboflavin), | |
| | Vitamin B-6 (Pyridoxine), Vitamin | |
| | B-12 (Cyanocobalamin), Natural Vitamin | |
| | (Acetate), Niacin, Biotin, Pantothenic | |
| | Acid, Zinc, Folic Acid, Vitamin K, | |
| | Selenium. Other Ingredients: Protein | |
| | Blend (Soy protein isolate, Hydrolyzed | |
| | collagen, Whey protein isolate, | |
| | Calcium/Sodium Caseinate), Glycerine, | |
| | Polydextrose (fiber), Water, Cocoa Butter, | |
| | Natural Coconut Oil (non-hydronated), | |
| | Coconut, Cellulose, Cocoa Powder, Olive | |
| | Oil, Lecithin, Natural and Artificial | |
| | Flavor, Maltodextrin, Guar Gum, | |
| | Citric Acid (Flavor Enhancer), | |
| | Sucralose | |

I. CEREAL WITH THE INVENTIVE COMPLEX

| Ingre- | | |
|---|---|---|
| dient | | Quantity per |
| No. | Ingredient | 1 Kg |
| 1 | Chromium (from Inventive Complex) | 50.00-500.00 mcg |
| 2 | Excipients: Whole Grain Oats, Oat Bran, | q.s |
| | Sugar, Modified Corn Starch, Brown | |
| | Sugar Syrup, Salt, Calcium Carbonate, | |
| | Trisodium Phosphate, Wheat Flour, | |
| | Vitamin E (Mixed tocopherols), Zinc & | |
| | Iron (Mineral nutrients), Niacinamide | |
| | (A B Vitamins), Vitamin B6 (Pyridoxine | |
| | Hcl), Vitamin B2 (Riboflavin), Vitamin | |
| | B1 (Thiamin Mononitrate), Vitamin A | |
| | (Palmitate), Vitamin A B (Folic acid), | |
| | Vitamin B12, Vitamin D | |

J. BEVERAGE WITH THE INVENTIVE COMPLEX

| Ingre- | | |
|---|---|---|
| dient | | Quantity per |
| No. | Ingredient | 500 mL |
| 1 | Chromium (from Inventive Complex) | 50.00-500.00 mcg |
| 2 | Excipients: Filtered Water, Food Starch- | q.s |
| | Modified, Citric Acid, Bitter Orange, | |
| | Green Tea Extract, Maltodextrin, Whey | |
| | Protein Isolate, High Fructose Corn | |
| | Syrup and/or Sucrose and/or Sugar, | |
| | Sodium Benzoate, Caffeine, Niacin, | |
| | Glycerol Ester of Wood resin, | |
| | Flavors, Colors | |

### EXAMPLE 4

### Animal Studies of Phenolic Antioxidant-Chromium Complexes

The following examples are included to illustrate certain preferred embodiments and aspects of the present invention and are not construed as limiting the scope thereof.

A. Screening of Antidiabetic Activity (Animal Studies)

| | |
|---|---|
| 1. Protocol | |
| Diabetic model | Steptozocine(STZ)-induced male |
| | albino rats |
| Number of animals | 5 |
| Number of groups | 6 |
| Treatment schedule | 3 days before and 2 days after STZ |
| | injection |
| Dose of STZ | 50 mg/Kg, intra venous |
| Dose and route of test drugs | 1 ml/Kg body weight, oral |
| Vehicle used to suspend test drugs | Arabic gum (2%) |
| Blood glucose estimation time | 48 hr after STZ injection, 18 hr |
| | fasting condition |
| Principal of glucose estimation | GOD/POD method (kit: Monozyme |
| | Ind. Ltd), Described by Tiez, In |
| | Clinical Guide to Laboratory tests, pp |
| | 238-240, 1976, W.B. saunders, |
| | Philadelphia, USA |
| STZ | Sigma-Aldrich |

2. Results - Blood Sugar Lowering Action of the Test Drugs

TABLE 1

| Treatment | Number of | Fasting Serum | |
|---|---|---|---|
| Group* | Animals | Glucose (mg/dl) (mean ± SEM) | % Inhibition |
| STZ | 5 | 338.48 ± 14.89 | — |
| N-1 + STZ | 5 | 262.36 ± 12.68 | 22.48 |
| N-2 + STZ | 5 | 243.88 ± 35.36 | 27.94 |
| N-3 + STZ | 5 | 139.06 ± 9.31 | 58.92 |
| N-5 + STZ | 5 | 170.24 ± 13.25 | 49.70 |
| N-7 + STZ | 4 | 141.30 ± 9.54 | 58.25 |

| *Description of the test drugs | Dosage |
|---|---|
| N-1 | Example - 1A, Phyllanthus emblica |
| | extract, 10 mg/Kg of the animal |
| | body weight |
| N-2 | Example - 1A, Phyllanthus emblica |
| | extract, 20 mg/Kg of the animal |
| | body weight |
| N-3 | Example - 2, Phyllanthus emblica |
| | extract, 10 mg/Kg plus Cr³⁺, 40 μg/Kg |
| | of the animal body weight |
| N-5 | Example 2, Phyllanthus emblica |
| | extract, 10 mg/Kg plus purified Shilajit |
| | plus Cr³⁺, 40 μg/Kg of the animal body |
| | weight |
| N-7 | Chromium polynicotinate, 300 μg/Kg |
| | of the animal body weight |

B. Effect of high doses of the inventive complex on euglycemic rats

| | |
|---|---|
| 1. Protocol | |
| Diabetic model | Steptozocine(STZ)-induced male |
| | Wistar rats |
| Number of animals | 5 |
| Treatment schedule | Administered concurrently in STZ- |
| | treated animals for 21 days |
| Dose of STZ | 40 mg/Kg, sub cutanous |
| Dose and route of test drugs | 1 ml/Kg body weight, oral |
| Vehicle used to suspend test drugs | Arabic gum (2%) |
| Blood glucose estimation time | Assessed on days 7, 14 and 21 |
| | following STZ administration |
| Principal of glucose estimation | GOD/POD method (kit: Monozyme |
| | Ind. Ltd), Described by Tiez, In |
| | Clinical Guide to Laboratory Tests, |
| | pp 238-240, 1976, W.B. Saunders, |
| | Philadelphia, USA |
| STZ | Sigma-Aldrich |
| 2. Results | |

TABLE 2

| | Number | | | | |
|---|---|---|---|---|---|
| Treatment | of | Blood Sugar Values (Arterial Blood) in Days | | | |
| Group* | Animals | 1 | 7 | 14 | 21 |
| STZ- | 5 | 78.4 ± | 102.0 ± 4.1 | 155.0 ± 6.4 | 218.5 ± 8.8 |
| treated | | 3.5 | | | |
| S-1 | 5 | — | 80.2 ± 4.3 | 78.8 ± 3.1 | 85.3 ± 80 |
| S-2 | 5 | — | 80.0 ± 1.8 | 84.9 ± 2.8 | 80.6 ± 3.7 |
| Chromium | 5 | — | 88.7 ± 4.4 | 94.3 ± 7.2 | 92.5 ± 3.5 |
| picolinate | | | | | |

| | |
|---|---|
| *Description of the Test Drugs | Dosages |

| | |
|---|---|
| S-1 | Example 1A, Phyllanthus emblica |
| | extract, 50 mg/Kg, Cr³⁺, 500 μg/Kg |
| | of the animal body weight |
| S-2 | Example 1A, Phyllanthus emblica |
| | extract, 100 mg/Kg, Cr³⁺, 1,000 μg/Kg |
| | of the animal body weight |

| | |
|---|---|
| Chromium picolinate | 100 mg/Kg, Cr3⁺, 700 μg/Kg of the |
| | animal body weight |

Phyllanthus emblica - Cr³⁺ complexes (present invention) had no
perceptible per se effect on blood sugar in euglycemic rats.
Effects on Animal Body Weight
Phyllanthus emblica - Cr³⁺ complexes of invention (S-2) had a profound
protective effect on the loss of body weight due to STZ-induced
hyperglycemia (see Table 3 below):

TABLE 3

| | Number | | | | |
|---|---|---|---|---|---|
| Treatment | of | Body Weight in gm, in Days | | | |
| Group | Animals | 1 | 7 | 14 | 21 |
| Vehicle control | 5 | 162 ± 4 | 176 ± 5 | 192 ± 7 | 208 ± 12 |
| STZ-treated | 5 | 168 ± 5 | 179 ± 6 | 164 ± 7 | 170 ± 7 |
| S-2 | 5 | 160 ± 6 | 182 ± 5 | 188 ± 7 | 204 ± 9 |
| Chromium | 5 | 166 ± 8 | 180 ± 7 | 168 ± 9 | 172 ± 9 |
| picolinate | | | | | |
| Example-1A, | 5 | 162 ± 8 | 168 ± 7 | 182 ± 8 | 198 ± 5 |
| 100 mg/Kg | | | | | |
| Example-1D, | 5 | 166 ± 5 | 190 ± 9 | 188 ± 11 | 206 ± 7 |
| 100 mg/Kg | | | | | |

C. Repairing Damaged β-Cells by the Test Compounds of the Present Invention

In another set of experiments, STZ-induced diabetic rats, after 21 days, were administered the invention test complex compounds: S-1 (50 mg/Kg, p.o.), S-2 (100 mg/Kg, p.o.), Chromium picolinate (100 mg/Kg, p.o.), Example-1A (100 mg/Kg., p.o.), per day for further 21 days. This experiment was conducted with a view to assessing the repair aspect of the damaged β-cells by the test compounds. The results are given in Table 4 below:

TABLE 4

| Treatment on | % Change | Glucose (mg/dl) values; mean ± SE, in Days | | | |
|---|---|---|---|---|---|
| Diabetic Rats | from day 1 | 1 | 7 | 14 | 21 |
| Vehicle control | +16.9 ↑ | 269 ± 16.1 | 333.4 ± 16.7 | 307.9 ± 21.57 | 315.3 ± 20.5 |
| (distilled water) | | | | | |
| S-1 | −8.5 ↓ | 296.0 ± 4.1 | 383.3 ± 8.1 | 286.6 ± 3.6 | 270.8 ± 5.7 |
| S-2 | −14.75 ↓ | 307.5 ± 7.0 | 303.7 ± 5.6 | 282.5 ± 5.9 | 262.1 ± 4.5 |
| Chromium | +14 ↑ | 253.2 ± 7.7 | 302.8 ± 18.0 | 294.3 ± 20.4 | 289.6 ± 9.0 |
| picolinate | | | | | |
| Example-1A, | +8.2 ↑ | 265.9 ± 6.8 | 268.4 ± 11.4 | 272.5 ± 7.8 | 288.0 ± 11.1 |
| 100 mg/Kg | | | | | |

Progressive increase in hyperglycemia (post STZ-treatment) was reversed, dose-dependently, by the Phyllanthus emblica—Cr³⁺ complex test sample (present invention). Phyllanthus emblica, as such, may thwart the rate of increase in blood glucose levels, while chromium picolinate had no beneficial effect on this parameter.

While the invention has been described with particular reference to certain embodiments thereof, it will be understood that changes and modifications may be made which are within the skill of the art. Accordingly, it is intended to be bound only by the following claims, in which:

1. A composition for the treatment, prevention or management of a condition in primates, especially humans comprising a phenolic antioxidant-chromium complex.

2. The composition of claim 1 wherein the condition is Type 2 diabetes or non-insulin dependent diabetes mellitus.

3. The composition of claim 1 wherein the condition is glucose intolerance.

4. The composition of claim 1 comprising a phenolic antioxidant having no pro-oxidation activity.

5. The composition of claim 1 wherein the phenolic antioxidant is of plant origin.

6. The composition of claim 1 wherein the chromium content in the complex is 0.01 to 20% of the complex.

7. The composition of claim 6 wherein the chromium content in the complex is from 0.02 to 10%.

8. The composition of claim 1 wherein the chromium is trivalent in nature.

9. The composition of claim 1 wherein the phenolic antioxidants include low molecular weight hydrolyzable tannins having a molecular weight below 2,000.

10. The composition of claim 9 wherein the phenolic antioxidant is obtained from the genus Phyllanthus, Terminalia, Gardenia, Geranium, Erodium or Tamarix.

11. The composition of claim 9 wherein the hydrolyzable tannins are obtained from Phyllanthus emblica (syn. Emblica officinalis), Phyllanthus amarus, Phyllanthus flexusus, other Phyllanthus species, Terminalia bellerica and other Terminalia species, Erodium pelagonium, Geranium thumbergi, Tamarix aphyla or another Tamarix species.

12. The composition of claim 11 wherein the condition in primates, especially humans is Type 2 diabetes or glucose intolerance.

13. The composition of claim 11 wherein the hydrolyzable tannins are obtained from the Phyllanthus emblica fruit.

14. A composition of claim 1 comprising chromium complex(s) of oxygenated dibenzo-α-pyrone (DBP) or its conjugates, including dimers and oligomers and fulvic acids for the treatment, prevention or management of Type 2 diabetes or glucose tolerance in primates, especially humans.

15. The composition of claim 14 wherein the oxygenated dibenzo-α-pyrone (DBP) or its conjugates, including dimers and oligomers and fulvic acids are obtained from purified Shilajit.

16. A composition of claim 1 comprising chromium complex(s) of the antioxidant fractions of Phyllanthus emblica and/or purified Shilajit, for the treatment, prevention or management of Type 2 diabetes or glucose intolerance.

17. The composition of claim 1 wherein the phenolic antioxidant-chromium complex is prepared by reacting a trivalent chromium salt with a phenolic antioxidant(s).

18. The composition of claim 17 wherein the phenolic antioxidant-chromium complex is prepared by reacting chromium chloride, acetate or formate with a phenolic antioxidant(s) in an aqueous system.

19. The composition of claim 18 wherein the phenolic antioxidant-chromium complex is prepared by reacting chromium chloride, acetate or formate with low molecular weight tannins having a molecular weight below 2,000.

20. The composition of claim 17 wherein the phenolic antioxidant-chromium complex is prepared by reacting chromium chloride, acetate or formate with oxygenated dibenzo-α-pyrone (DBP) or its conjugates, including dimers and oligomers and fulvic acids of purified Shilajit in an aqueous system.

21. The composition of claim 17 wherein the phenolic antioxidant-chromium complex is obtained by spray, freeze, tray or vacuum drying.

22. A formulation of the composition of claim 1 wherein the phenolic antioxidant-chromium complex is combined with a pharmaceutically or nutritionally acceptable excipient.

23. A formulation of claim 22 wherein the phenolic antioxidant-chromium complex is combined with a pharmaceutically or nutritionally acceptable excipient for the treatment of Type 2 diabetes or glucose intolerance in primates, especially humans.

24. The composition of claim 1 which also includes an added active ingredient.

25. The composition of claim 24 wherein said added active ingredient is an antioxidant, vitamin, carnitine, carnosine, N-acetyl-L-cysteine, biotin, polycosanol, aminoguanidine, a fatty acid or plant extract, or mixtures thereof.

26. The composition of claim 7 wherein the chromium content in the complex is 1 to 8% of the complex.

27. The composition of claim 19 wherein the molecular weight of said tannins is below 1,000.

28. A method of treating, preventing or managing a condition in primates, especially humans which comprises treating said primate, especially human with the composition of claim 1.

29. A method of claim 28 wherein said condition is Type 2 diabetes or glucose intolerance.

30. A formulation of claim 22 wherein the phenolic antioxidant-chromium complex having 10 to 1,000 μg of chromium content is combined with a pharmaceutically or nutritionally acceptable excipient to improve insulin sensitivity, reduce blood glucose, glycated hemoglobin, reduce total cholesterol and low density lipids in primates, especially humans.

31. The composition of claim 1 wherein the phenolic antioxidant-chromium complex is prepared by dry blending a trivalent chromium salt or a complex with a phenolic antioxidant(s).

32. The composition of claim 31 wherein the phenolic antioxidant-chromium complex is prepared by dry blending chromium chloride, acetate or formate, picolinate, nicotinate or polynicotinate with a phenolic antioxidant(s).

33. The composition of claim 31 wherein the phenolic antioxidant-chromium complex is prepared by dry blending chromium chloride, acetate, formate, nicotinate, polynicotinate or picolinate with oxygenated dibenzo-α-pyrone (DBP) or its conjugates, including dimmers and oligomers and fulvic acids of purified Shilajit.

34. A formulation of claim 31 wherein the phenolic antioxidant-chromium blend having 10 to 1,000 μg of chromium is combined with a pharmaceutically or nutritionally acceptable excipient to improve Type 2 diabetes, glucose intolerance, insulin sensitivity, reduce blood glucose, glycated hemoglobin, reduce total cholesterol and low density lipid in primates, especially humans.

35. A pharmaceutical or nutritional preparation of claim 34 is administered once or twice a day to a primate, especially human.
